(19) United States
(12) Patent Application Publication
(10) Pub. No.: US 2011/0094520 A1
(43) Pub. Date: Apr. 28, 2011

(54) SYSTEM AND METHOD FOR TREATMENT OF SNORING

(76) Inventors: EUGENE MIKHAILENOK, San Diego, CA (US); Olga Voronina, San Diego, CA (US)

(21) Appl. No.: 12/913,628

(22) Filed: Oct. 27, 2010

Related U.S. Application Data
(63) Continuation-in-part of application No. 10/974,304, filed on Oct. 27, 2004, now Pat. No. 7,801,887.

(51) Int. Cl. A61F 5/56

(52) U.S. Cl. 128/848

(57) ABSTRACT

A system and method for abatement to snoring and snoring disorders which tones and strengthens soft palate muscles is disclosed. The method comprises inserting a tube into one's mouth; pressing the tube up to the hard palate; holding the tube in the mouth at the hard palate using the tongue; and initiating a sucking process over the tube. The sucking process requires the person to maintain constant suction on the tube member for between about 5 to 15 seconds followed by a relaxation for between about 5 to 20 seconds and repeating this process of suction, hold, and relaxation for between about 10 to 20 times. A system provided by the present invention provides tubes of varying diameters and liquids of different viscosities.

### PRIORITY CLAIM

This utility patent application contains subject matter claiming benefit of the priority date of copending U.S. patent application Ser. No. 10/974,304, filed on Oct. 27, 2004, entitled, “Method and Device for Strengthening Soft Palate Muscles,” accordingly, the entire contents of this utility patent application is hereby expressly incorporated by reference.

### BACKGROUND OF THE INVENTION

This present invention relates to an improvement in methods for strengthening or toning of soft palate muscles as an abatement to snoring and snoring disorders, and more particularly to a unique method for such strengthening and toning and a specific tool suited to facilitate such strengthening and toning.

Snoring is a common problem among many people; especially the aged. Referring to FIG. 1, simply put, snoring by a person 110 is a sound generated by the soft tissues of the nasopharynx 20 as air, incoming and outgoing, passes through the nasal ducts 15 and further through the nasal cavity 16 over the hard palate 17, or through the mouth 12 and the oral cavity 13 under the hard palate 17, when lower jaw 11 droops during sleep.

Two particular groups of problems related to snoring for the snoring party are: [1] social-esthetical and [2] medical. As for the first, the snoring party can be annoying to a bed partner and, depending upon how loud the sound, how frequent, and how long, can cast the snoring party into an unflattering view.

The most important of these problems, however, are medical, such as multiform sleep disorders and life threatening obstructive apnea [the temporary cessation of breathing]. The cause of snoring can be due to anatomic defects of the nasopharynx (e.g., nasal stenosis, an elongated lingula 19 of the soft palate, and the like). The most widespread cause of snoring, however, is due to a weakness, or weaker tonicity, of the soft palate muscles.

The soft palate is an anatomic part of the nasopharynx which aids one in the functions of swallowing, in suction, and in phonation. It basically is a muscle-cartilage slab, located dorsally of the hard palate. The thin muscle layer which is basic material composing the soft palate and which dorsally extends past it, forms the lingula. These muscle segments [i.e., the soft palate and the lingula are referred to as the soft palate muscles] are relatively strong and well toned in children and young adults. Such strength and tonicity generally prevents snoring. For that reason, snoring is relatively rare in children and young adults.

But as one ages, the tonicity of muscles of the soft palate become less toned and weaker. As one then sleeps supine, or on their backside, the weakened soft palate muscles often do not prevent a sagging of the soft palate which then lies more loosely and down toward the nasopharynx. In such a condition, as air passes by, because the soft palate muscles are not as toned or as strong as before, it may flap and vibrate; and snoring often results. It is estimated that more than 60% of people over 50 snore while sleeping. Approximately 10% of these snorers also suffer from apnea, the most clinically dangerous form of snoring (referred to as obstructive apnea).

Current treatments for mild forms of snoring include the following:

a. changing one's position while sleeping (not sleeping in the supine position but rather making a conscious effort to sleep only on one's side or in a prone position);

b. the use of special pillows shaped to control head position; and

c. engaging in pharmacological remedies, such as peels and sprays, which are designed to prevent excessive moisture concentration and congestion in the nasopharynx.

Though well suited for their intended purpose of minimizing snoring or preventing it, these treatments are temporary in nature and attack and treat only the symptoms of snoring but not the primary cause of it. As a result, these methods and treatments are limited in their effectiveness.

For more severe forms of snoring, the following treatments are typical:

a. masks or other passive appliances (such as gears and other apparatus) for displacing the lower jaw and moving it forward thus opening the nasopharynx to create a better clearance for the airflow as one sleeps; and

b. active mech that generate a permanent high-pressure flow of air in and through the airways as one sleeps [known as Continuous Positive Airway Pressure (CPAP)] therapy.

These technologies, as much as the previously described ones, also do not attack a primary cause of snoring; i.e., weakened soft palate muscles. Because these treatments are symptomatic only, those being treated with these regimens must continue to use them for the duration of their lives. These treatments are cumbersome and uncomfortable. Primarily because of the discomfort associated with such treatments and that they are treatments of the symptoms rather than the causes of snoring, more than 20% of the persons targeted for these regimens have refused them or, after having tried them, have discontinued their use.

The most effective regimen involves surgery of which several surgical procedures were developed. These include:

a. cryogenic or laser treatment of the palate tissues. Such treatment creates scar tissue to the treated palate region. The tissue thereby thickens and becomes tougher or harder which results in an alteration of its resonance characteristics;

b. resectioning of the lingua and a brim of the soft palate; and

c. more complicated surgeries that increase the nasopharynx clearance.

Surgical regimens are more intrusive, more costly, and, of the more complex nasopharynx surgery, more dangerous. As with all forms of surgery, other complications, such as, but not limited to narcosis and blood loss, may result. Other less serious consequences often result from surgical procedures of this nature. These may include snuffling voice, swallowing difficulties and the internal mis-direction of ingested food into the nasal ducts and nasopharynx rather than into the esophagus during the swallowing process. Therefore, surgical intervention should be employed only in and for life-threatening situations.

The present invention is a non-invasive manner of addressing and correcting a root cause of snoring, is simple in approach and execution, and is more permanent in nature than the prior art approaches, devices, and methods of treatment.

As such, the present invention is based on a pathogenetical approach to the abatement of snoring and, as such, is directed at a major cause of the snoring, weak soft palate muscles 18 and comprises a non-automated tool. The tool of the present invention strengthens and reinforces the soft palate muscles 18 by means of a specialized and regimented training program specific to these muscles which requires repeated and progressive active contraction of the soft palate muscles 18 which, by following this process, over time, causes a gradual increase in mass and firmness of the soft palate muscles 18 and results in the following benefits:

a. strengthening and toning the soft palate muscles which has the effect of it being more horizontally disposed which thereby increases the nasopharynx clearance 23 and, by such clearance, decreases or limits any resistance to airflow; and

b. with such strengthening and toning, the present invention changes the resonance characteristics of the soft palate and thereby prevents or reduces sound oscillations caused by interaction between those tissues and the incoming and outgoing airflow.

The suggested tool of the present invention for the strengthening of soft palate muscles for the abatement of snoring has an important distinction from all currently existing methods, procedures, and devices, which at best are temporary and at worst, are invasive and cumbersome. A soft palate and associated muscles that are trained, toned, and strengthened through use of the present invention retain their high tonicity and strength for a longer period of time after the course of training has concluded. This in turn provides for a longer-term desired result for the person. Maintaining the regimen of the present invention will yield even greater, if not more permanent positive results.

The foregoing has outlined some of the more pertinent objects of the present invention. These objects should be construed to be merely illustrative of some of the more prominent features and applications of the intended invention. Many other beneficial results can be attained by applying the disclosed invention in a different manner or by modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### BRIEF SUMMARY OF THE INVENTION

The above-noted problems, among others, are overcome by the present invention. Briefly stated, the present invention contemplates a system and method for abatement to snoring and snoring disorders which calls for training to tone and strengthen soft palate muscles by inserting a tube member into one's mouth; pressing the tube member up to the hard palate; holding the tube member in the mouth at the hard palate using the tongue; and initiating a sucking process over the tube member. The sucking process requires the person to maintain constant suction on the tube member for between about 5 to 15 seconds followed by a relaxation for between about 5 to 20 seconds and repetition of this process of suction, hold, and relaxation for between about 10 to 20 times.

More specifically in a first aspect, the invention may be characterized as a method for strengthening of soft palate muscles as an abatement to snoring and snoring disorders comprising: (a) providing a tube member of a predetermined diameter, the tube member having a first open end, a second open end, and a passageway there between; (b) inserting the tube member first open end into a user's mouth; (c) pressing the first end of said tube member up to a hard palate in the user's mouth; (d) holding the tube member in the user's mouth with a tongue of the user; (e) inserting said second end into a container having a liquid therein; and (f) initiating a sucking process over said tube member to begin removing said liquid from said container. Additional method steps include (g) maintaining a contraction phase to initiate constant suction on said tube member for between about 5 to 15 seconds followed by; (h) maintaining a relaxation phase of disengaging from said contraction phase for between about 5 to 20 seconds, wherein step (g) and step (h) further completes a cycle, followed by; and (i) repeating step (g) and step (h) until said liquid is removed from said container, wherein step (g) and step (h) together with the repeating completes a session and establishes a drain time.

The invention in this aspect further comprises establishing a first drain time, wherein the first drain time is a reference drain time. Additionally, the method includes engaging in not less than four (4) sessions per week and between about twenty (20) to fifty five (55) sessions. Still further method steps include replacing the tube member with a smaller diameter tube member having a smaller diameter by approximately between 5% to 25% smaller, wherein the drain time for the session decreases by between about 15% to 25%; and returning a current session drain time to the reference drain time.

The invention in this aspect is yet further characterized in that the replacing the tube member is performed after approximately every 2 to 4 sessions. Also, the liquid is characterized as being approximately 330 milliliters and having a viscosity of between about 1,000 centi-Poise to about 10,000 centi-Poise. Still further, the method herein calls for discontinuing the method upon abatement of snoring; and resuming the method upon snoring reoccurring. Lastly, the method in this aspect is characterized wherein pressing the first end of said tube member up to the hard palate in the user's mouth comprises placing the tube member approximately two inches into a mouth up to the hard palate as to not engage cheek muscles while not inducing a gag reflex.

In a second aspect, the present invention is a system for snoring abatement comprising a first tube member of a predetermined diameter, the first tube member having a first open end, a second open end, and a passageway there between, wherein the first tube member first open end is inserted into a user's mouth and pressed up to a hard palate in the user's mouth and with a tongue sealing the tube, and wherein sucking is initiated to strength the muscles of the soft palate, thereby providing abatement from snoring. The system further comprises a container having a first liquid, wherein the sucking is initiated to the first liquid in the container; a plurality of second and subsequent tube members having decreasing diameters with respect to the first tube member; and a plurality of second and subsequent liquids having increasing viscosities with respect to the first liquid.

The foregoing has outlined the more pertinent and important features of the present invention in order that the detailed description of the invention that follows may be better understood so the present contributions to the art may be more fully appreciated. Additional features of the present invention will be described hereinafter which form the subject of the claims. It should be appreciated by those skilled in the art that the conception and the disclosed specific embodiment may be readily utilized as a basis for modifying or designing other structures and methods for carrying out the same purposes of the present invention. It also should be realized by those skilled in the art that such equivalent constructions and methods do not depart from the spirit and scope of the inventions as set forth in the appended claims.

### BRIEF DESCRIPTION OF THE DRAWINGS

For a fuller understanding of the nature and objects of the invention, reference should be had to the following detailed description taken in conjunction with the accompanying drawings in which:

FIG. 1 is detailed cross-sectional side view of a person's head.

FIG. 2 is a schematic diagram of the snoring abatement tool.

### DETAILED DESCRIPTION OF THE INVENTION

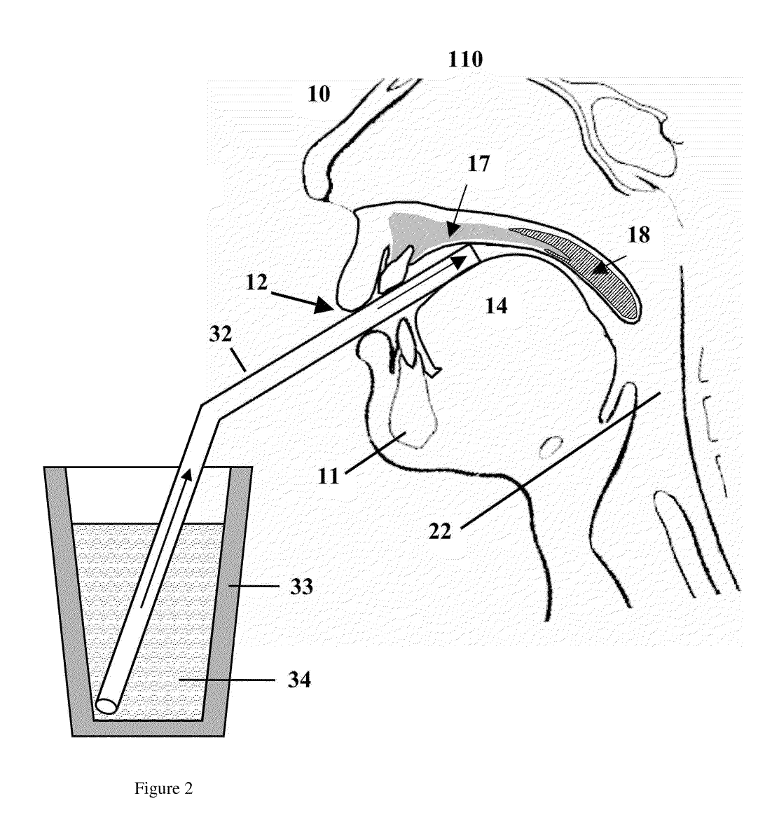

In the normal course of sucking liquid from a container through a straw 32 [see FIG. 2] or similar tube-like or elongated object, several muscles are at play and work synchronously with one another [for the purpose of discussion only and not by means of limitation and unless otherwise indicated, the term straw or tube will be used for such tube-like objects].

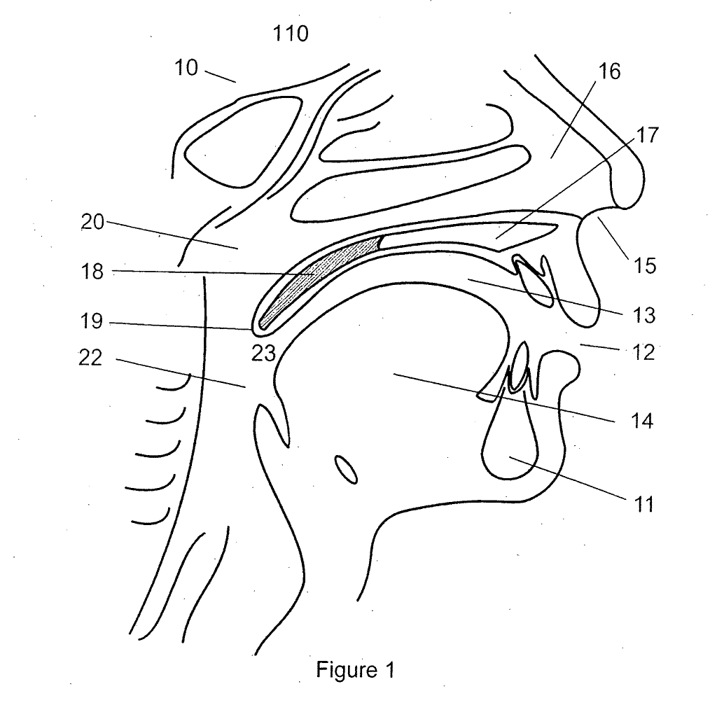

Referring to FIG. 1, reference character 110 represents a person and reference character 10 represents the detailed cross-section of the person's head so as to better illustrate the body parts/muscles at play while engaging in suction efforts through a straw 32. Generally, these are the muscles of tongue 14, cheeks, and soft palate muscles 18. To initiate a sucking process, the muscles engage in a “work-out” by contracting. An important object of the present invention is to direct the process of contraction only to the soft palate muscles 18 such that generally these muscles are the muscles being trained and to exclude the influence other muscles have in the sucking process by minimizing, if not preventing, their contraction. Placement of the straw 32 into the mouth 12 of a person is important to the process of the present invention.

Reference is now made also to FIG. 2. In this illustration, the upper end of the tube 32 is to be placed into the person's mouth 12 approximately 2 inches into the mouth past the lips, pushed up to the hard palate 17 and held there with the tongue 14. Two inches is used herein for ease of explanation. What is necessary is to place the tube 32 far enough into the mouth onto the hard palate 17 so as not to engage the cheek muscles by not placing the tube 32 far enough in and not so far in that the tube 32 induces vomiting. Placement of the tube 32 into the hard palate 17 between approximately 45% to approximately 75% of the full distance of the hard palate 17 is generally sufficient and, in most people will equate to approximately or at least 2 inches.

Placement and retention of the straw 32 by the tongue 14 at the hard palate 17, according to the invention, alters the natural involvement of muscles in the suction process. More specifically, the cheek muscles do not participate in the suction and involvement of the root of the tongue 14 [radix linguae] is minimal; and the main suction force is provided by the soft palate muscles 18. In these conditions a submaximal contraction of the soft palate muscles becomes feasible, which is an ultimate condition for increasing mass and strength during successive training sessions. Conversely, placement and retention of the straw 32 in a typical manner (retention by lips or teeth) causes insufficient contraction of the soft palate muscles and therefore can not lead to increase in their strength and mass.

In a preferred embodiment, no tool other than a straw 32, a container 33, and [if used] a liquid 34 are required for this training. If used, the liquid should be of a relatively high viscosity and is placed into a container. After the straw is placed in the mouth 12 as described above, the other end of the straw 32 is placed into the liquid 34. The person is then instructed to engage in the training sessions [Se]. A training session entails repeated cycles [Cy] of:

(1) sucking the liquid 34 through the straw 32 [the contraction phase CtPh)]; and

(2) resting [i.e., not sucking the liquid 34 through the straw 32, the relaxation phase (RxPh)].

Further in the preferred embodiment, there should be about 10 to 20 such cycles [Cy]. Usually 10-20 cycles are sufficient to make the muscles tired enough; which is a criterion of successful training. The contraction phase [CtPh] should be continuous and typically should last for between about 5 to 15 seconds [though between about 7-10 seconds will generally yield optimal results] followed by the relaxation phase [RxPh] of between about 5 to 20 seconds [though between about 10-15 seconds will yield positive results]. The person should engage in maximum or near to maximum, effort during the contraction phase [CtPh]. The duration of a typical session [Se] generally should be between about 5 to 15 minutes [though about 10 minutes will yield positive results]. Typically there should be no more than two sessions per day, not less than four sessions per week, and between about 20 to 55 [or more] sessions overall. Optimal results, however, generally are realized after the completion of the 35th session.

As indicated above, a liquid need not be used for such training sessions. Instead, after proper placement of the straw 32 as described above, a person may merely suck on any solid elongate object or a hollow elongate object having no end cap openings. In essence, the person would be sucking on an object which offers resistance and thereby exercises [engages in the contraction phase of] the desired muscle group. The sessions as described above would generally be the same and, if properly supervised, would realize the desired results of strengthening and toning the soft palate muscles 18. Use of a suitable liquid 34 with a straw 32, however, facilitates the training, adds a pleasant and nutritional feature [ingestion] to the training, and may be conducted without medical supervision.

Where a liquid 34 is used, its viscosity and the diameter of the straw 32 should be such that, following the session process described above, approximately 330 ml. of liquid would be sucked from the container in approximately between about 5 to 15 minutes [drain time]. For a liquid having the viscosity of a dietary product known as UltraSlim® [approximately 1,200 centi-Poise] and using a straw 32 having a diameter of between about 2 to 8 mm. [preferably between about 3 to 4 mm.], a person should be able to suck the 330 ml. from the container between that time frame.

Thus the viscosity of a liquid used for this purpose should be between about 1,000 centi-Poise [cP] to about 10000 cP which is approximately equivalent to an interval of viscosity between that of pancake syrup and honey. As a soaked-up liquid it is possible to use other less viscous liquids or water (which has a viscosity of approximately 1000 cP), but in such cases it will be necessary to use tubes having extremely small internal diameters (0.1-0.3 mm).

Not every person will have the same contraction attributes and stamina as to maintain a continuous draw on the straw 32 for the same amount of time as other persons. The time it takes one person to suck all the liquid from the container [drain time] may typically vary from person to person. Therefore, at the first session, a person's reference point [RfPt], should be established. A person's reference point [RfPt] in this embodiment is the amount of time it takes the person to suck all the liquid from the container following the process described above. That period of time is referred to as the reference time [RfTm]. Therefore, for this embodiment a person's RfPt=RfTm.

With each succeeding session of continued and progressive contraction of the soft palate muscle 18, this muscle group will become stronger and more and more toned. The time it takes a person to suck out the liquid from the container [drain time] will, thereby, begin and continue to decrease. At that the moment that the person's current drain time decreases by between approximately 15-25% from the reference drain time [RfTm], using the same liquid at subsequent sessions, the original straw bearing the original diameter should replaced with a replacement straw of a smaller diameter [replacement diameter]. For optimum results, typically a 20% decrease in RfTm warrants a replacement straw.

The diameter of the replacement straw should be sufficiently smaller as to return the current session drain time to approximately the reference point drain time. Generally this translates to the replacement straw replacement diameter being approximately between about 5% to 25% smaller that the original straw's original diameter. In addition to replacing the original straw as drain time decreases as described above, the original straw should also be replaced after between about every 2 to 4 sessions. Such straw replacement [by decreasing drain time and/or by sessions] ensures a progressive increase in effort during the contraction phase [CtPh] by the person with concomitant favorable results in strengthening and toning the soft palate muscles 18.

As described above, approximately 35 such sessions are generally the optimum for a person 110 undergoing this regimen in order to attain a sufficiently high degree of tonicity as to significantly reduce and, in most cases, eliminate the act of snoring. Approximately 35 sessions also are generally the optimum in order to maintain that high level of tonicity for a greater period of time thereby permitting the person to discontinue the training sessions until, and if, snoring recurs. If, when, snoring recurs, the person should resume the training and toning of the soft palate muscle 18 in the manner and duration described above.

The present disclosure includes that contained in the present claims as well as that of the foregoing description. Although this invention has been described in its preferred forms with a certain degree of particularity, it is understood that the present disclosure of the preferred forms has been made only by way of example and numerous changes in the details of construction and combination and arrangement of parts and method steps may be resorted to without departing from the spirit and scope of the invention. Accordingly, the scope of the invention should be determined not by the embodiment[s] illustrated, but by the appended claims and their legal equivalents.

The invention claimed is:

1. A method for strengthening of soft palate muscles as an abatement to snoring and snoring disorders comprising:
(a) providing a tube member of a predetermined diameter, the tube member having a first open end, a second open end, and a passageway there between;
(b) inserting the tube member first open end into a user's mouth;
(c) pressing the first end of said tube member up to a hard palate in the user's mouth;
(d) holding the tube member in the user's mouth with a tongue of the user;
(e) inserting said second end into a container having a liquid therein; and
(f) initiating a sucking process over said tube member to begin removing said liquid from said container.

2. The method of claim 1 wherein said sucking process comprises:
(g) maintaining a contraction phase to initiate constant suction on said tube member for between about 5 to 15 seconds followed by;
(h) maintaining a relaxation phase of disengaging from said contraction phase for between about 5 to 20 seconds, wherein step (g) and step (h) further completes a cycle, followed by; and
(i) repeating step (g) and step (h) until said liquid is removed from said container, wherein step (g) and step (h) together with the repeating completes a session and establishes a drain time.

3. The method of claim 2 further comprising establishing a first drain time, wherein the first drain time is a reference drain time.

4. The method of claim 2 further comprising engaging in not less than four (4) sessions per week.

5. The method of claim 2 further comprising engaging in between about twenty (20) to fifty five (55) sessions.

6. The method of claim 3 further comprising:
replacing said tube member with a smaller diameter tube member having a smaller diameter by approximately between 5% to 25% smaller, wherein the drain time for the session decreases by between about 15% to 25%; and
returning a current session drain time to the reference drain time.

7. The method of claim 6, wherein the replacing is performed after approximately every 2 to 4 sessions.

8. The method of claim 1, said liquid comprising:
approximately 330 milliliters; and
a viscosity of between about 1,000 centi-Poise to about 10,000 centi-Poise.

9. The method of claim 1 further comprising:
discontinuing the method upon abatement of snoring; and
resuming the method upon snoring reoccurring.

10. The method of claim 1, wherein pressing the first end of said tube member up to the hard palate in the user's mouth comprises placing the tube member approximately two inches into a mouth up to the hard palate as to not engage cheek muscles while not inducing a gag reflex.

11. A system for snoring abatement comprising a first tube member of a predetermined diameter, the first tube member having a first open end, a second open end, and a passageway there between, wherein the first tube member first open end is inserted into a user's mouth and pressed up to a hard palate in the user's mouth, and wherein sucking is initiated to strength the muscles of the soft palate, thereby providing abatement from snoring.

12. The system of claim 11, further comprising:
a container having a first liquid, wherein the sucking is initiated to the first liquid in the container;
a plurality of second and subsequent tube members having decreasing diameters with respect to the first tube member; and
a plurality of second and subsequent liquids having increasing viscosities with respect to the first liquid.
